(19) United States
(12) Patent Application Publication
(10) Pub. No.: US 2004/0092485 A1
(43) Pub. Date: May 13, 2004

(54) Ascorbic acid 2-phosphate metal salt with low calcium content

(76) Inventors: Yoshio Fujiwara, Kawasaki-shi Kanagawa (JP); Yuji Kobayashi, Kawasaki-shi Kanagawa (JP); Makoto Saito, Kawasaki-shi Kanagawa (JP)

Correspondence Address: SUGHRUE MION, PLLC, 2100 PENNSYLVANIA AVENUE, N.W., SUITE 800, WASHINGTON, DC, 20037, US

(21) Appl. No.: 10/469,527

(22) PCT Filed: Mar. 1, 2002
(86) PCT No.: PCT/JP02/01926

(30) Foreign Application Priority Data
Mar. 2, 2001 (JP) 2001-57982

(51) Int. Cl. A61K 31/665

(52) U.S. Cl. 514/99; 549/216

(57) ABSTRACT

One of the object of the present invention is to provide an L-ascorbic acid 2-phosphate metal salt, which causes no clouding and scarcely precipitates or deposits even when added to a cosmetic material or the like having blended therein an organic acid.

Disclosed herein is an L-ascorbic acid-2-phosphate metal salt with a low calcium content characterized in that the content of a calcium compound is 500 ppm or less in terms of calcium ion, and production process thereof.

### CROSS REFERENCES OF RELATED APPLICATION

This application is an application filed under 35 U.S.C.§111(a) claiming benefit pursuant to 35 U.S.C. §119(e) (1) of the filing date of Provisional Application 60/275,033 filed on Mar. 13, 2001 pursuant to 35 U.S.C. §111(b).

### DETAILED DESCRIPTION OF THE INVENTION

1. Application Field in Industry

The present invention relates to L-ascorbic acid derivative reduced in the calcium content and a production process therefor. More specifically, the present invention relates to L-ascorbic acid 2-phosphate metal salt having a low calcium content, which is useful as an additive for cosmetic, medical, food and feed materials, widely applicable in various industrial fields and particularly when added to a cosmetic material containing an organic acid, causes no clouding or scarcely precipitates or deposits even in the storage of cosmetic material, and also relates to a production process therefor.

2. Background Art

The L-ascorbic acid (vitamin C) has been heretofore used in various fields such as cosmetics, medical preparations, food and feed because of its various physiological and pharmacological activities such as suppression of lipoperoxide, acceleration of collagen formation, retardation of melanin formation and enhancement of immunity function.

However, L-ascorbic acid is known to be unstable to oxygen, heat, light and the like and readily undergo coloration or degradation. Therefore, L-ascorbic acid generally used in the field of cosmetic material is not L-ascorbic acid itself but a derivative improved in the stability against oxygen, heat and the like by forming the hydroxyl group at the 2-position of L-ascorbic acid into a phosphoric ester or a phosphate salt. Among these derivatives, L-ascorbic acid-2-phosphate metal salts, particularly magnesium salt, are widely used as a vitamin C derivative having excellent stability and being easily soluble in water.

There are know a lot of processes for producing L-ascorbic acid 2-phospate metal salts. As for the process for producing such L-ascorbic acid 2-phosphate magnesium salt (hereinafter sometimes referred to as “APM”), for example, a method of reacting L-ascorbic acid-2-phosphate (hereinafter sometimes referred to as “2-AP”) with magnesium oxide, magnesium hydroxide, magnesium carbonate or the like to produce L-ascorbic acid 2-phosphate magnesium salt is known. This production process of APM is described in a large number of publications such as JP-B-52-18191 (the term “JP-B” as used herein means an “examined Japanese patent publication”), JP-A-59-51293 (the term “JP-A” as used herein means an “unexamined published Japanese patent application”) and JP-A-2-286693.

However, the thus-obtained APM has a problem in that when blended, for example, in a cosmetic material such as cosmetic lotion, it readily causes clouding, precipitation or deposition during storage. Aggregation of ingredients in the production process of APM has been considered as one of causes therefor. More specifically, in the production of APM, when APM solution is crystallized in a poor solvent such as methanol or dried in a vacuum, aggregation readily occurs and the particle size increases. If this APM increased in the particle size is blended in a cosmetic material, clouding, precipitation or the like is readily caused due to alcohols contained in the cosmetic material.

In order to prevent such clouding, various methods have been heretofore proposed, for example, a method of blending citrate, oxalate, gluconate or alanine in the above-described cosmetic material to a high concentration (see, JP-A-1-213212, JP-A-4-283593), a method of freeze-drying the obtained APM before use (see, JP-A-2-231496), or a method of spray-drying the obtained APM before use (see, JP-A-7-112914).

Blending of citrate, oxalate, gluconate or alanine in a high concentration only can attain slight retardation of the precipitation or deposition of APM and although the precipitation or deposition of APM may be slightly improved by the use of freeze-dried or spray-dried APM, the effect is not sufficiently high.

The problems associated with precipitation or deposition are not only specific in APM but also found in various -ascorbic acid 2-phosphate metal salts.

### OBJECT OF THE INVENTION

The present invention has been made to solve the above-described problems and one of the object of the present invention is to provide L-ascorbic acid 2-phosphate metal salt having a low calcium content, which causes no clouding and scarcely precipitates or deposits even when added to a cosmetic material or the like having blended therein an organic acid. This object of the present invention includes providing a production process therefor.

Another object of the present invention is to provide a cosmetic material, a medical preparation, a beverage, a food and a feed, each containing L-ascorbic acid 2-phosphate metal salt and being free from generation of clouding, precipitation or deposition.

### SUMMARY OF THE INVENTION

As a result of extensive investigations to solve the above-described problems, the present inventors have newly found this time that the clouding generated, in a cosmetic material during storage of the cosmetic material containing L-ascorbic acid 2-phosphate metal salt is caused by not only the L-ascorbic acid 2-phosphate metal salt deposited due to alcohols contained in the cosmetic material but also by salts produced in the reaction between an organic acid (for example, citric acid) present in the cosmetic material and calcium contained as an impurity in the starting material L-ascorbic acid 2-phosphate metal salt; that clouding is readily generated when this L-ascorbic acid 2-phosphate metal salt has a high calcium content; that the calcium originates in a metal compound used as a starting material; and that when the content of calcium contained as an impurity in the starting metal compound is reduced, a cosmetic lotion or the like free from generation of clouding can be obtained. The present invention has been accomplished based on these findings.

More specifically, the present invention relates to the following matters.

(1) An L-ascorbic acid-2-phosphate metal salt with a low calcium content characterized in that the content of a calcium compound is 500 ppm or less in terms of calcium ion.

(2) The L-ascorbic acid-2-phosphate metal salt with a low calcium content as described in (1), wherein the L-ascorbic acid-2-phosphate metal salt is a salt of alkaline earth metal other than calcium, alkali metal salt, zinc salt, aluminum salt or titanium salt.

(3) The L-ascorbic acid-2-phosphate metal salt with a low calcium content as described in (1), wherein the L-ascorbic acid-2-phosphate metal salt is sodium salt, potassium salt, magnesium salt, zinc salt or aluminum salt.

(4) The L-ascorbic acid-2-phosphate metal salt with a low calcium content as described in (1), wherein the L-ascorbic acid-2-phosphate metal salt is potassium salt, magnesium salt or zinc salt.

(5) The L-ascorbic acid-2-phosphate metal salt with a low calcium content as described in (1), wherein the L-ascorbic acid-2-phosphate metal salt is magnesium salt.

(6) A process for producing an L-ascorbic acid-2-phosphate metal salt with a low calcium content wherein the content of a calcium compound is 500 ppm or less in terms of calcium ion, said process is characterized by adding a metal compound containing a calcium compound in an amount of 2,000 ppm or less in terms of calcium ion to a solution containing an L-ascorbic acid-2-phosphoric ester.

(7) The process for producing an L-ascorbic acid-2-phosphate metal salt with a low calcium content as described in (6), wherein the metal compound is any one of potassium compound, sodium compound, magnesium compound or zinc compound, and the resulting L-ascorbic acid-2-phosphate metal salt is any one of potassium salt, sodium salt, magnesium salt or zinc salt.

(8) The process for producing an L-ascorbic acid-2-phosphate metal salt with a low calcium content as described in (6), wherein the metal compound is any one of magnesium oxide or magnesium hydroxide, and the resulting L-ascorbic acid-2-phosphate metal salt is magnesium salt.

(9) The process for producing an L-ascorbic acid-2-phosphate metal salt with a low calcium content as described in any one of (6) to (8), wherein the solution containing an L-ascorbic acid-2-phosphoric ester contains alkali metal ion or alkaline earth metal ion in an amount of 10 ppm or less in terms of the L-ascorbic acid-2-phosphoric ester.

(10) The process for producing an L-ascorbic acid-2-phosphate metal salt with a low calcium content as described in (9), wherein the solution containing an L-ascorbic acid-2-phosphoric ester contains alkali metal ion or alkaline earth metal ion in an amount of 1 ppm or less in terms of the L-ascorbic acid-2-phosphoric ester.

(11) The process for producing an L-ascorbic acid-2-phosphate metal salt with a low calcium content as described in (9) or (10), wherein the solution containing an L-ascorbic acid-2-phosphoric ester is prepared by a method comprising reacting L-ascorbic acid, an alkali metal hydroxide and phosphorylating agent in the presence of water, and removing metal ion from the resulting solution of L-ascorbic acid-2-phosphate metal salt by using ion exchange resin.

(12) The process for producing an L-ascorbic acid-2-phosphate metal salt with a low calcium content as described in (11), wherein the ion exchange resin is weakly or moderately basic anion-exchange resin.

(13) A low-clouding cosmetic material characterized in that the L-ascorbic acid 2-phosphate metal salt with a low calcium content described in (1) is blended.

(14) The low-clouding cosmetic material described in (13), wherein the cosmetic material is lotion.

(15) The low-clouding cosmetic material described in (13) or (14), wherein 0.1 to 10% by weight of organic acid is blended in the cosmetic material.

(16) A low-clouding medical preparation characterized in that the L-ascorbic acid 2-phosphate metal salt with a low calcium content described in (1) is blended.

(17) A low-clouding beverage characterized in that the L-ascorbic acid 2-phosphate metal salt with a low calcium content described in (1) is blended.

Specific Description of the Invention

The L-ascorbic acid 2-phosphate metal salt with a low calcium content according to the present invention and the production process therefor are described in detail below.

L-ascorbic Acid 2-Phosphate Metal Salt with Low Calcium Content

The high-purity L-ascorbic acid 2-phosphate metal salt with a low calcium content according to the present invention is characterized in that the content of calcium compound as an impurity is 500 ppm or less in terms of calcium ion. Herein, the L-ascorbic acid-2-phosphate metal salt is a salt of metal other than calcium, preferably a salt of alkaline earth metal other than calcium, alkali metal salt, zinc salt, aluminum salt or titanium salt, more preferably sodium salt, potassium salt, magnesium salt, zinc salt or aluminum salt, still preferably potassium salt, magnesium salt or zinc salt, and most preferably magnesium salt.

Such the L-ascorbic acid 2-phosphate metal salt causes no clouding and scarcely precipitates or deposits even when added to a cosmetic lotion containing an organic acid such as citric acid, or ethanol. Accordingly, when such the L-ascorbic acid 2-phosphate metal salt is blended, various cosmetic, medical preparations, food and feed materials having excellent storage stability can be obtained.

In the highly purified L-ascorbic acid 2-phosphate metal salt with a low calcium content according to the present invention, the content of calcium compound as an impurity is suitably 500 ppm or less, preferably 300 ppm or less, in terms of calcium ion. If the calcium compound content exceeds 500 ppm, when the L-ascorbic acid 2-phosphate metal salt is blended in a cosmetic material or the like, a salt is formed with an organic acid (for example, a citric acid) contained in the cosmetic material or the like to readily cause clouding, precipitation or deposition.

Production of L-Ascorbic Acid 2-Phosphate Metal Salt with Low Calcium Content

In a preferred embodiment of the process for producing an L-ascorbic acid 2-phosphate metal salt with a low calcium content according to the present invention, a metal compound containing an impurity calcium compound in the later described amount or less is added to a solution containing an L-ascorbic acid-2-phosphoric ester (2-AP) (this solution is hereinafter sometimes called “a 2-AP-containing solution”). Herein, the metal compound is a compound of a metal other than calcium, preferably a compound of alkaline earth metal other than calcium, alkali metal compound, zinc compound, aluminum compound or titanium compound, more preferably sodium compound, potassium compound, magnesium compound, zinc compound or aluminum compound, still preferably potassium compound, sodium compound, magnesium compound or zinc compound, and most preferably magnesium compound.

By adding such the metal compounds, the finally obtained L-ascorbic acid 2-phosphate metal salt can be reduced in the content of calcium compound as an impurity and by using this L-ascorbic acid 2-phosphate metal salt reduced in the calcium content, a cosmetic lotion or a medical or food material can be prevented from clouding or the like.

In the present invention, the metal compound may be added to a 2-AP-containing solution and the adding method is not particularly limited insofar as the 2-AP and the metal compound can be contacted and reacted.

In producing APM by, for example, adding a metal compound to a 2-AP-containing solution and thereby reacting 2-AP with the metal compound, a commonly known method described in JP-B-52-18191, JP-A-59-51293, JP-A-2-286693 and the like may be employed.

More specifically, the above-described reaction may be performed by adding a metal compound such as metal oxide having a calcium content in a fixed amount or less to a 2-AP-containing solution, adjusting the pH after the addition to 8.5 to 10.5, and allowing the solution to stand at a temperature of 5 to 25° C. for 3 to 5 hours.

2-AP-Containing Solution

The 2-AP-containing solution (starting material solution) for use in the present invention is not particularly limited and, for example, the following solutions may be used.

Examples of the 2-AP-containing solution which can be suitably used include a 2-AP-containing solution obtained by directly phosphorylating an ascorbic acid (see, JP-B-43-9219, JP-B-45-23746 and JP-A-6-345786) and a 2-AP-containing solution obtained by phosphorylating a 5,6-O-isopropylidene-L-ascorbic acid (see, JP-B-43-9219, JP-B-45-4497, JP-B-45-30328 and JP-B-59-4438) may be preferably used. In addition, a 2-AP-containing solution produced from an L-ascorbic acid and a phosphoric acid donor under the action of an enzyme or a microorganism (see, JP-A-2-42996) may also be used.

More specifically, the 2-AP-containing solution can be obtained, for example, by a method where an L-ascorbic acid, an alkali metal hydroxide and a phosphorylating agent are reacted in the presence of water at a pH of 6 to 13, preferably from 6 to 8, and the obtained 2-AP alkali metal salt solution (an alkali metal salt solution of L-ascorbic acid-2-phosphoric ester) is treated with an ion exchange resin to remove metal ion.

Still more specifically, the 2-AP-containing solution can be obtained, for example, by a method where an L-ascorbic acid is dissolved in a mixed solvent of pyridine and water, reacted with a phosphorylating agent such as phosphorus oxychloride while adjusting the pH to 6 to 13, preferably 6 to 8, using an aqueous alkali metal hydroxide solution (for example, an aqueous solution of sodium hydroxide, potassium hydroxide or lithium hydroxide) and at the same time, with the alkali metal hydroxide, and the obtained 2-AP alkali metal salt solution is treated with an ion exchange resin to remove metal ion.

In the present invention, a 2-AP alkali metal salt solution or a 2-AP alkaline earth metal salt solution (an alkaline earth metal salt solution of L-ascorbic acid-2-phosphoric ester) may be used in place of the 2-AP-containing solution. In this case, these solutions (aqueous solution) are preferably subjected to a metal ion-removing treatment (hereinafter sometimes referred to as decationization) before use. Examples of the decationization method include a method of treating a 2-AP metal salt solution with an appropriate ion exchange resin.

More specifically, for example, a 2-AP metal salt solution such as L-ascorbic acid 2-phosphate magnesium salt (APM) is passed through a strongly acidic cation exchange resin and then water is eluted, whereby the cation portion (metal ion) of the 2-AP metal salt solution is exchanged with hydrogen ion, and the metal ion remains on the exchange resin, as a result, a decationized 2-AP solution is obtained. The 2-AP-containing solution can also be obtained by a general method, for example, where a 2-AP metal salt solution (aqueous solution) is passed through a weakly or moderately basic anion-exchange resin to adsorb 2-AP to the ion exchange resin and at the same time, allow metal ion (cation) such as alkali metal ion or alkaline earth metal ion to flow out, and the adsorbed 2-AP is eluted with an acid such as 0.1 to 2 N dilute hydrochloric acid.

In the present invention, the thus-obtained high-purity 2-AP extremely reduced in the metal ion content may be used, where the content of alkali metal ion or alkaline earth metal ion, such as Na ion or Ca ion, is usually 10 ppm or less, preferably 1 ppm or less in terms of the L-ascorbic acid-2-phosphoric ester.

Metal Compound

The metal compound which is used as a starting material in the production process of the L-ascorbic acid 2-phosphate metal salt according to the present invention is, as mentioned above, a compound of a metal other than calcium, preferably a compound of alkaline earth metal other than calcium, alkali metal compound, zinc compound, aluminum compound or titanium compound, more preferably sodium compound, potassium compound, magnesium compound, zinc compound or aluminum compound, still preferably potassium compound, sodium compound, magnesium compound or zinc compound, and most preferably magnesium compound. The metal compound is, for instance, metal oxide, metal hydroxide or metal carbonate, and preferably metal oxide or metal hydroxide. Accordingly, examples of magnesium compound include, for instance, magnesium oxide, magnesium hydroxide and magnesium carbonate. Among these, magnesium oxide and magnesium hydroxide are preferred.

The content of calcium compounds as an impurity contained in the metal compound for use in the present invention is suitably 2,000 ppm or less, preferably 1,500 ppm or less, more preferably 750 ppm or less, in terms of calcium ion. If the calcium content in the starting material metal salt exceeds 2,000 ppm, the L-ascorbic acid 2-phosphate metal salt produced using the metal compound has a high calcium concentration and when blended in a cosmetic material or the like, an organic acid (e.g., citric acid) contained in the base material of the cosmetic material reacts with the calcium compound to form a salt, as a result, clouding, precipitation or deposition is readily caused and this is not preferred.

When 2-AP and the metal compound are reacted as such by adding a metal compound having an impurity calcium compound content of 2,000 ppm or less in terms of calcium ion to a 2-AP-containing solution, the obtained L-ascorbic acid 2-phosphate metal salt can have a low calcium content of 500 ppm or less, preferably 300 ppm or less, in terms of calcium ion.

Uses of L-ascorbic Acid 2-phosphate Metal Salt with Low Calcium Content

The L-ascorbic acid 2-phosphate metal salt with a low calcium content according to the present invention does not cause clouding, precipitation or deposition even when added to a cosmetic material having blended therein particularly an organic acid (e.g., citric acid, succinic acid, malic acid, tartaric acid) and stored for a long period of time, and therefore, can be suitably used in various cosmetic materials.

Examples of the cosmetic material to which the L-ascorbic acid 2-phosphate metal salt with low calcium content obtained by the present invention can be added include:

a lotion where citric acid, sodium citrate, ε-aminocaproic acid, 1,3-butylene glycol, ethanol, glycerol, polyoxyethylene hydrogenated castor oil, purified water, perfume and the like are blended;

a cream where stearic acid, cetanol, 1,3-butylene glycol, parahydroxybenzoic ester, dipentaerythritol fatty ester, purified water, perfume and the like are blended;

a milky lotion where stearic acid, cetanol, octyldodecyl erucate, polyoxyethylene tetraoleate and the like are blended; and

a pack where dipropylene glycol, sodium pyrrolidone-carboxylate, polyvinyl alcohol, tetrasodium edetate and the like are blended. However, the present invention is not limited thereto.

The ratio of L-ascorbic acid 2-phosphate metal salt with low calcium content blended to these cosmetic materials varies depending on the amount of organic acid blended, the preparation form and the like but assuming that the entire amount of the cosmetic material is 100 wt %, the ratio is generally from 0.01 to 30 wt %, preferably from 0.1 to 20 wt % in a lotion; generally from 0.01 to 30 wt %, preferably from 0.1 to 15 wt % in a cream; generally from 0.01 to 30 wt %, preferably from 0.1 to 20 wt % in a milky lotion; and generally from 0.01 to 30 wt %, preferably from 0.1 to 15 wt % in a pack. The amount of the organic acid contained in the cosmetic material varies depending on the kind of the cosmetic material but is generally from 0.1 to 10 wt %.

This L-ascorbic acid 2-phosphate metal salt reduced in the calcium content can be suitably used also for medical preparations (e.g., preparation for oral cavity, ophthalmic solution, bath preparation), beverages, feed for animals, food and the like containing particularly an organic acid (e.g., citric acid, succinic acid, malic acid, tartaric acid) and the same effect as in the cosmetic material can be obtained.

### EXAMPLES

The present invention is described in greater detail below by referring to examples, however, the present invention is not limited to these Examples. In the following examples, the invention is described in accordance with the production of L-ascorbic acid 2-phosphate magnesium salt. However, as mentioned above, the invention is not limited to magnesium salt, but include the various metal salts of L-ascorbic acid 2-phosphate metal.

### Example 1

In 368 ml of pure water, 32 g of L-ascorbic acid 2-phosphate magnesium salt was dissolved. The resulting solution was passed through a column packed with 2,000 ml of a strongly acidic cation exchange resin (Amberlite IR-120B, produced by Organo) and further through 1,200 ml of pure water to obtain 1,600 ml of an aqueous solution containing only 2-AP. The magnesium ion content of this solution was less than 1 ppm and the content of calcium compound as an impurity was lower than the detection limit.

While keeping this aqueous 2-AP solution at 10° C., magnesium hydroxide having an impurity calcium compound content of 500 ppm in terms of calcium ion was added to give a pH of 9.2 after the addition. The resulting solution was left standing for 4 hours and the supernatant was filtered to remove insoluble matters. The filtrate solution as concentrated to one-tenth (1/10) and after adding a two-fold amount of methanol, the generated solid portion was collected by filtration. Subsequently, the obtained solid portion (wet body) was recrystallized with water-methanol and then dried to obtain an L-ascorbic acid 2-phosphate magnesium salt as a white dry product. The calcium compound content in this white dry product was 120 ppm in terms of calcium ion. Using this dry product, in-solution stability tests 1 and 2 described later were performed, as a result, clouding was not observed.

The concentration of calcium compound as an impurity in the starting material magnesium hydroxide and in the final product L-ascorbic acid 2-phosphate magnesium salt was measured by ICP (manufactured by Seiko Instruments K.K.) (similarly measured in Example 2 and Comparative Examples 1 to 2 below).

### Comparative Example 1

An L-ascorbic acid 2-phosphate magnesium salt as a white dry product was obtained in the same manner as in Example 1 except that magnesium hydroxide having an impurity calcium compound content of 2,500 ppm in terms of calcium ion was used in place of the magnesium hydroxide having an impurity calcium compound content of 500 ppm in terms of calcium ion in Example 1. The calcium compound content in this white dry product was 580 ppm. Using this dry product, in-solution stability tests 1 and 2 described later were performed. In both in-solution stability tests 1 and 2, clouding was observed at room temperature (20° C.) and more distinguished clouding was observed at 50° C.

### Example 2

In a nitrogen atmosphere, 100 g of L-ascorbic acid was added to and dissolved in a mixed solvent containing 1,350 ml of pure water and 150 g of pyridine and after cooling to 0 to 10° C., an aqueous 10% sodium hydroxide solution was added to adjust the pH to about 12. To the resulting solution, 150 g of phosphorus oxychloride was added dropwise and while adjusting the pH to 12 with an aqueous 10% sodium hydroxide solution, a reaction was performed by keeping the temperature at 0 to 10° C. After the completion of dropwise addition, the pH was adjusted to about 7 with 35% hydrochloric acid and pyridine was distilled off under reduced pressure. Thereafter, 35% hydrochloric acid was added to adjust the pH to 4.

The reaction solution after such adjustment of pH was diluted by adding 6,500 ml of pure water, passed through a column packed with 2,000 ml of a moderately basic anion exchange resin (Amberlite IRA-68, produced by Organo) and then developed with 23,500 ml of 0.05N hydrochloric acid and further with 11,000 ml of 0.2N hydrochloric acid to obtain a fraction containing 2-AP.

An aqueous solution of this fraction was dialyzed using an electrodialyser (Model DU-Ob, manufactured by Asahi Glass K.K.) until chlorine ion was reduced to 500 ppm. While keeping the dialysate solution at 15° C., magnesium oxide having an impurity calcium compound content of 1,000 ppm in terms of calcium ion was added to give a pH of 10.1 after the addition. This solution was left standing for 2 hours and the supernatant was filtered to remove insoluble matters. The resulting solution was concentrated to one-tenth (1/10) and after adding a two-fold amount of methanol, the generated solid portion was collected by filtration. The obtained wet body was recrystallized with water-methanol and then dried to obtain an L-ascorbic acid 2-phosphate magnesium salt as a white dry product. The impurity calcium compound content in this white dry product was 280 ppm in terms of calcium ion. Using this dry product, in-solution stability tests 1 and 2 described later were performed, as a result, clouding was not observed.

### Comparative Example 2

An L-ascorbic acid 2-phosphate magnesium salt as a white dry product was obtained in the same manner as in Example 2 except that magnesium oxide having an impurity calcium compound content of 3,000 ppm in terms of calcium ion was used in place of the magnesium oxide having an impurity calcium compound content of 1,000 ppm in terms of calcium ion in Example 2. The calcium compound content in this white dry product was 750 ppm in terms of calcium ion. Using this dry product, in-liquid stability tests described later were performed. As a result, in the in-solution stability test 1, clouding was observed when the dry product was allowed to stand at room temperature, and more distinguished clouding was observed when allowed to stand at 50° C. In the in-liquid stability test 2, distinguished clouding was observed under both conditions of room temperature and 50° C.

In-solution Stability Test 1

Into a 200 ml-volume Erlenmeyer flask, 2 g of a sample (APM), 0.4 g of sodium citrate dihydrate and 0.05 g of citric acid were charged. Thereto, 16 g of glycerol·1,3-butylene glycol·ethanol mixed solution (volume ratio: 3:5:8) was added and the resulting solution was stirred for 30 minutes. Thereto, 82 g of pure water was added and the resulting solution was further stirred for 2 hours. After filtering this solution through a filter, a half amount was left standing at room temperature (20° C.) for 1 month and the remaining half amount was left standing at 50° C. for 10 days. Each solution was observed with an eye on the clouding state.

The observation results are shown in Table 1.

In-solution Stability Test 2

Into a 200 ml-volume Erlenmeyer flask, 2 g of a sample (APM), 0.4 g of sodium citrate dihydrate and 0.05 g of citric acid were charged. Thereto, 15 g of glycerol·1,3-butylene glycol·ethanol mixed solution (volume ratio: 3:5:8) was charged and the resulting solution was stirred for 30 minutes. Thereto, 82 g of pure water was added and further stirred for 2 hours. After filtering this solution through a filter, a half amount was left standing at room temperature (20° C.) for 1 month and the remaining half amount was left standing at 50° C. for 10 days. Each solution was observed with an eye on the clouding state.

The observation results are shown in Table 1.

TABLE 1

| | |
|---|---|
| In-solution Stability | In-solution Stability |
| Test 1 | Test 2 |

| | Room | | Room | |
|---|---|---|---|---|
| | Temperature | | Temperature | |
| | (20° C.) | 50° C. | (20° C.) | 50° C. |
| Example 1 | A | A | A | A |
| Example 2 | A | A | A | A |
| Comparative | B | C | B | C |
| Example 1 | | | | |
| Comparative | B | C | C | C |
| Example 2 | | | | |

A: No clouding,
B: Clouded,
C: Distinguished Clouding

Effect of the Invention

According to the production process of the present invention, L-ascorbic acid 2-phosphate metal salt with a low calcium content can be produced, where the content of calcium compound as an impurity contained in the L-ascorbic acid 2-phosphate metal salt, giving rise to clouding, precipitation or deposition, is reduced to 500 ppm or less in terms of calcium ion. The L-ascorbic acid 2-phosphate metal salt with a low calcium content according to the present invention can be free of clouding, precipitation or deposition during storage either at room temperature or at a high temperature (for example, 50° C.) when the L-ascorbic acid 2-phosphate metal salt is blended in a cosmetic material containing particularly an organic acid, and therefore, can be suitably used for this usage. Furthermore, this L-ascorbic acid 2-phosphate metal salt can exhibit stability during the same storage as above even when blended in a medical, feed or food material containing particularly an organic acid, and therefore, can be widely used in various industrial fields.

1. An L-ascorbic acid-2-phosphate metal salt with a low calcium content characterized in that the content of a calcium compound is 500 ppm or less in terms of calcium ion.

2. The L-ascorbic acid-2-phosphate metal salt with a low calcium content as claimed in claim 1, wherein the L-ascorbic acid-2-phosphate metal salt is a salt of alkaline earth metal other than calcium, alkali metal salt, zinc salt, aluminum salt or titanium salt.

3. The L-ascorbic acid-2-phosphate metal salt with a low calcium content as claimed in claim 1, wherein the L-ascorbic acid-2-phosphate metal salt is sodium salt, potassium salt, magnesium salt, zinc salt or aluminum salt.

4. The L-ascorbic acid-2-phosphate metal salt with a low calcium content as claimed in claim 1, wherein the L-ascorbic acid-2-phosphate metal salt is potassium salt, magnesium salt or zinc salt.

5. The L-ascorbic acid-2-phosphate metal salt with a low calcium content as claimed in claim 1, wherein the L-ascorbic acid-2-phosphate metal salt is magnesium salt.

6. A process for producing an L-ascorbic acid-2-phosphate metal salt with a low calcium content wherein the content of a calcium compound is 500 ppm or less in terms of calcium ion, said process is characterized by adding a metal compound containing a calcium compound in an amount of 2,000 ppm or less in terms of calcium ion to a solution containing an L-ascorbic acid-2-phosphoric ester.

7. The process for producing an L-ascorbic acid-2-phosphate metal salt with a low calcium content as claimed in claim 6, wherein the metal compound is any one of potassium compound, sodium compound, magnesium compound or zinc compound, and the resulting L-ascorbic acid-2-phosphate metal salt is any one of potassium salt, sodium salt, magnesium salt or zinc salt.

8. The process for producing an L-ascorbic acid-2-phosphate metal salt with a low calcium content as claimed in claim 6, wherein the metal compound is any one of magnesium oxide or magnesium hydroxide, and the resulting L-ascorbic acid-2-phosphate metal salt is magnesium salt.

9. The process for producing an L-ascorbic acid-2-phosphate metal salt with a low calcium content as claimed in any one of claims 6 to 8, wherein the solution containing an L-ascorbic acid-2-phosphoric ester contains alkali metal ion or alkaline earth metal ion in an amount of 10 ppm or less in terms of the L-ascorbic acid-2-phosphoric ester.

10. The process for producing an L-ascorbic acid-2-phosphate metal salt with a low calcium content as claimed in claim 9, wherein the solution containing an L-ascorbic acid-2-phosphoric ester contains alkali metal ion or alkaline earth metal ion in an amount of 1 ppm or less in terms of the L-ascorbic acid-2-phosphoric ester.

11. The process for producing an L-ascorbic acid-2-phosphate metal salt with a low calcium content as claimed in claim 9 or 10, wherein the solution containing an L-ascorbic acid-2-phosphoric ester is prepared by a method comprising reacting L-ascorbic acid, an alkali metal hydroxide and phosphorylating agent in the presence of water, and removing metal ion from the resulting solution of L-ascorbic acid-2-phosphate metal salt by using ion exchange resin.

12. The process for producing an L-ascorbic acid-2-phosphate metal salt with a low calcium content as claimed in claim 11, wherein the ion exchange resin is weakly or moderately basic anion-exchange resin.

13. A low-clouding cosmetic material characterized in that the L-ascorbic acid 2-phosphate metal salt with a low calcium content claimed in claim 1 is blended.

14. The low-clouding cosmetic material as claimed in claim 13, wherein the cosmetic material is lotion.

15. The low-clouding cosmetic material as claimed in claim 13 or 14, wherein 0.1 to 10% by weight of organic acid is blended in the cosmetic material.

16. A low-clouding medical preparation characterized in that the L-ascorbic acid 2-phosphate metal salt with a low calcium content claimed in claim 1 is blended.

17. A low-clouding beverage characterized in that the L-ascorbic acid 2-phosphate metal salt with a low calcium content claimed in claim 1 is blended.
